(19) United States
(12) Patent Application Publication
(10) Pub. No.: US 2003/0207448 A1
(43) Pub. Date: Nov. 6, 2003

(54) Methodologies for the creation of pluripotent or multipotent human stem cells without creating or destroying a human embryo

(76) Inventor: Gregory Henry Revera, Madison, AL (US)

Correspondence Address: Gregory H. Revera, 109 Pebble Brook Cir., Madison, AL, 35758, US

(21) Appl. No.: 10/430,966

(22) Filed: May 6, 2003

Related U.S. Application Data
(60) Provisional application No. 60/378,000, filed on May 6, 2002.

(51) Int. Cl. C12N 5/08; C12N 15/85

(52) U.S. Cl. 435/366; 435/455

(57) ABSTRACT

Methods to create human stem cells without creating or destroying a human embryo have been developed. The invention includes a method of creating a human stem cell by obtaining a non-human stem cell and removing the nucleus. The nucleus of a human donor cell is transferred to the non-human stem cell.

### CROSS-REFERENCE TO RELATED APPLICATIONS

Priority is claimed to United States Provisional Patent Application “Methodologies for the Creation of Pluripotent or Multipotent Human Stem Cells Without Creating or Destroying a Human Embryo” No. 60/378,000 filed on May 6, 2002.

### BACKGROUND OF INVENTION

1. Field of the Invention

The invention relates generally to methods for creating human stem cells.

2. Background Art

In the 1980s and 1990s advances were made in animal cloning technologies that made it possible to produce clones of an animal with desirable traits. This was accomplished by removing the nucleus of the oocyte of one animal and inserting the nucleus from any somatic cell of the animal one wished to clone.

This process, called nuclear transfer technology, was viewed as a possible improvement over traditional animal husbandry. Nuclear transfer methodologies have become well known in the art. One such method is cell fusion where an enucleated oocye is fused with a donor somatic cell by causing the membranes of the two cells to fuse. Another method is microinjection where the donor nucleus is directly injected into the enucleated oocyte.

As cloning technologies improved, research began to focus on the cells that form early in embryonic development. These unspecialized cells are termed stem cells. A stem cell is a special kind of cell that has a unique capacity to renew and also to give rise to specialized cell types. Although most cells of the body, such as heart cells or skin cells, are committed to conduct a specific function, a stem cell is uncommitted and remains uncommitted, until it receives a signal to develop into a specialized cell. The ability of stem cells to become almost all of the specialized cells of the body give them the potential to generate replacement cells for a broad array of tissues and organs, such as the heart, the pancreas, and the nervous system. Special attention has been focused on three types of stem cells, namely: totipotent, pluripotent and multipotent stem cells.

Totipotent cells have the capability of differentiating to any of the types of cells found in the subject organism. A fertilized egg is a totipotent cell, as from it come all cells found in the organism. The first couple of divisions of a fertilized egg also results in the creation of totipotent cells. After several divisions, the cells begin to differentiate and form a blastocyst. The inner cell mass of the blastocyst consists of cells that are pluripotent. Pluripotent cells are capable of differentiating to create many different cell types but not all. Individually they do not have the potential to form an embryo in vivo. The inner cell mass next develops into the hypoblast and the epiblast. The hypoblast cells do not give rise to the formation of the embryo's body or the tissues that eventually occur in the mature organism. However, the cells of the epiblast give rise to all the cells of the embryo's body and are therefore also pluripotent.

Further differentiation of pluripotent stem cells lead to multipotent stem cells. Multipotent stem cells are more specialized than pluripotent stem cells. Multipotent stem cells give rise to cells that have a particular function. For example, as shown in FIG. 1, a single hematopoietic stem cell gives rise to red blood cells, macrophages, platelets, etc. FIG. 2 is a diagram showing the natural differentiation of a fertilized egg to the various embryonic tissues. The diagram shows which cells in embryonic development become the embryonic tissues (part of the embryo's body) and which part become extraembryonic tissues (not part of the embryo's body). FIG. 3 shows the location in the gastrula (post-blastocyst embryo) of various multipotent cells and examples of specialized cells those multipotent cells may develop.

In recent years, special interest has been focused on human pluripotent and multipotent stem cells. Under proper culture conditions known in the art these cells can multiply and retain their pluripotent or multipotent nature. Under differing conditions known in the art these cells can be made to differentiate into the different and specific tissues found in the human body. These tissues can then be used for therapeutic and research purposes. Under current methodologies, human pluripotent stem cells and most multipotent stem cells are cultured by using and destroying a human embryo. Moral, ethical and legal considerations often limit the usefulness of these methodologies. A solution is therefore needed to produce Human pluripotent and multipotent stem cells without necessitating the creation or destruction of a human embryo.

### SUMMARY OF INVENTION

In some aspects, the present invention relates to a method of creating a human stem cell, comprising: transferring the nucleus of a human somatic donor cell into an enucleated non-human mammalian stem cell forming a nuclear transfer unit; and activating the nuclear transfer unit.

In other aspects, the present invention relates to a method of creating a human stem cell, comprising: enucleating a non-human mammalian stem cell; and transferring the nucleus of a human somatic donor cell into the enucleated non-human mammalian stem cell.

In other aspects the present invention relates to a method of creating human stem cells, comprising: enucleating a non-human mammalian stem cell; transferring the nucleus of a human somatic donor cell into the enucleated non-human mammalian stem cell forming a nuclear transfer unit; and activating the nuclear transfer unit.

In other aspects the present invention relates to a method of creating a human stem cell, comprising: enucleating a human oocyte; transferring the nucleus of a non-human mammalian somatic donor cell into the enucleated oocyte creating a first nuclear transfer unit; and activating the nuclear transfer unit.

In other aspects the present invention relates to a method of creating a human stem cell, comprising: enucleating a human oocyte; transferring the nucleus of a non-human mammalian somatic donor cell into the enucleated human oocyte creating a first nuclear transfer unit; activating the first nuclear transfer unit; culturing the first nuclear transfer unit to yield at least one non-human stem cell; enucleating the non-human stem cell; and transferring the nucleus of a human somatic donor cell into the enucleated non-human stem cell to create a second nuclear transfer unit.

In other aspects, the present invention relates to a method of creating a human stem cell, comprising: enucleating a human oocyte; transferring the nucleus of a non-human mammalian somatic donor cell into the enucleated human oocyte creating a first nuclear transfer unit; activating the first nuclear transfer unit; culturing the first nuclear transfer unit to yield at least one non-human stem cell; enucleating the non-human stem cell; transferring the nucleus of a human somatic donor cell into the enucleated non-human stem cell to create a second nuclear transfer unit; and activating the second nuclear transfer unit.

In other aspects, the present invention relates to a method of creating a human stem cell, comprising: step for transferring the nucleus of a human somatic donor cell into an enucleated non-human mammalian stem cell forming a nuclear transfer unit; and step for activating the nuclear transfer unit.

In other aspects, the present invention relates to a method of creating a human stem cell, comprising: step for enucleating a non-human mammalian stem cell; and step for transferring the nucleus of a human somatic donor cell into the enucleated non-human mammalian stem cell.

In other aspects, the present invention relates to a method of creating a human stem cell, comprising: step for enucleating a human oocyte; and step for transferring the nucleus of a non-human mammalian somatic donor cell into the enucleated human oocyte.

In other aspects, the present invention relates to a method of creating a nuclear transfer unit comprising: enucleating a non-human mammalian stem cell; and transferring the nucleus of a human somatic donor cell into the enucleated non-human mammalian stem cell.

In other aspects, the present invention relates to a method of creating a nuclear transfer unit comprising: step for enucleating a non-human mammalian stem cell; and step for transferring the nucleus of a human somatic donor cell into the enucleated non-human mammalian stem cell.

In other aspects, the present invention relates to a method of creating a nuclear transfer unit comprising: enucleating a human oocyte; and transferring the nucleus of a non-human mammalian somatic donor cell into the enucleated oocyte.

In other aspects, the present invention relates to a method of creating a nuclear transfer unit comprising: step for enucleating a human oocyte; and step for transferring the nucleus of a non-human mammalian somatic donor cell into the enucleated oocyte.

Advantages of the present invention include the ability to create human multipotent or pluripotent stem cells without creating or destroying a human embryo. The present invention also teaches how to create nuclear transfer units with human nuclear material without creating or destroying a human embryo.

Other aspects and advantages of the invention will be apparent from the following description and the appended claims.

### BRIEF DESCRIPTION OF DRAWINGS

FIG. 1 shows the prior art paths of differentiation of a single Hematopoietic stem cell.

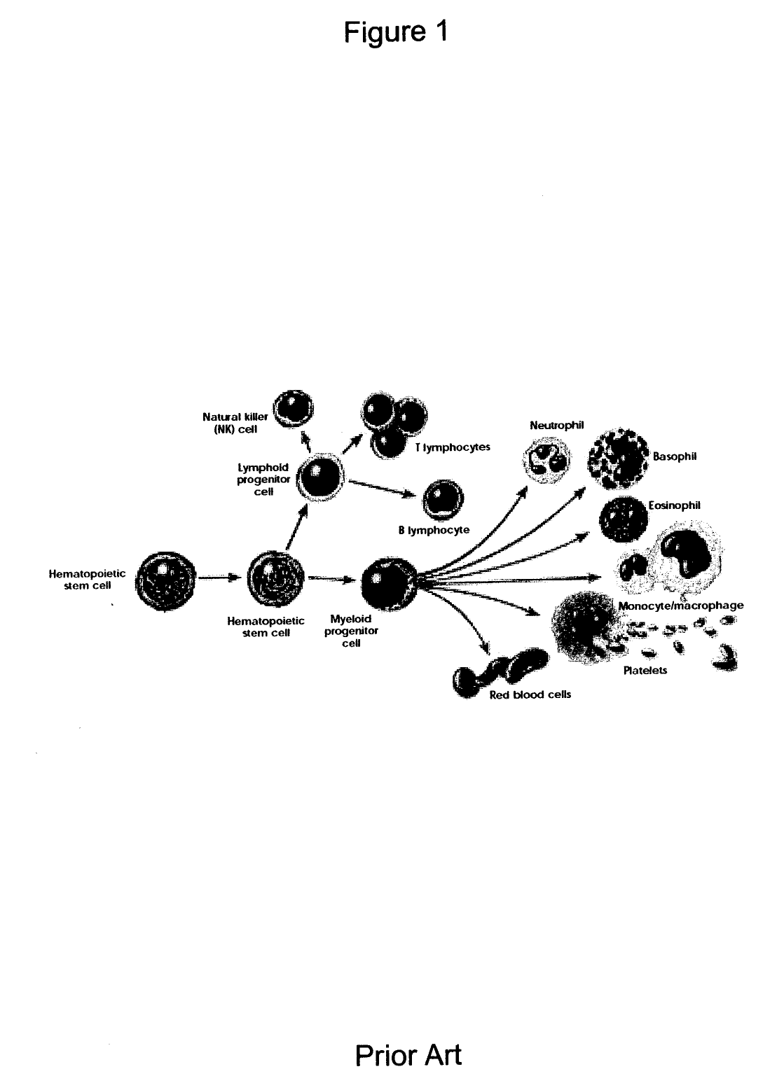

FIG. 2 diagrams the prior art differentiation of a fertilized egg into various embryonic and extraembryonic tissues of an embryo.

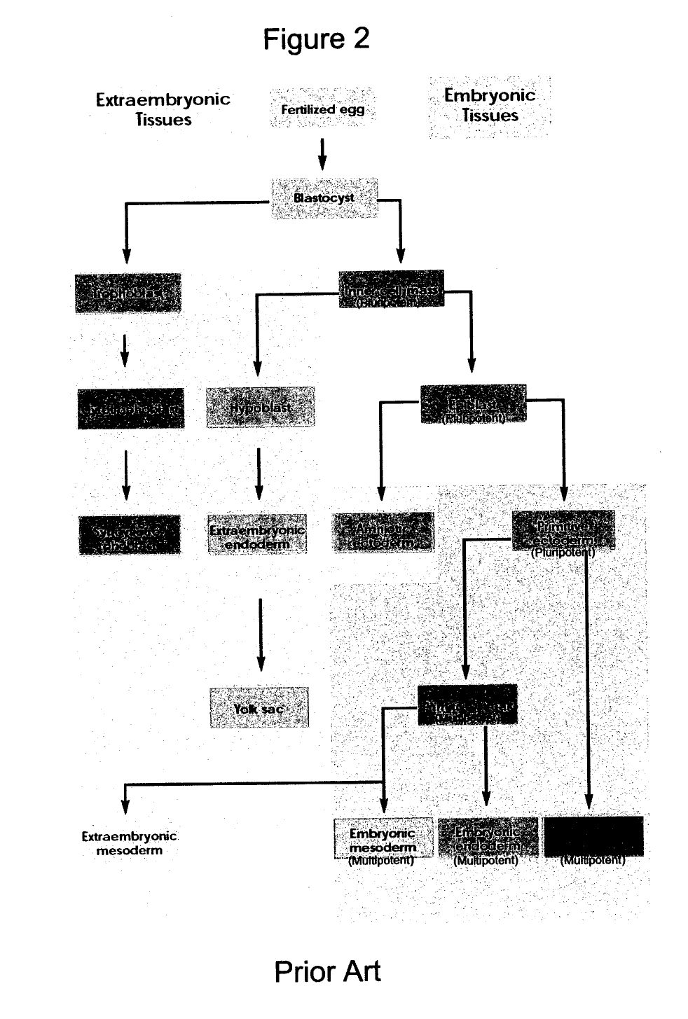

FIG. 3 shows the prior art location in the gastrula of various multipotent cells and examples of specialized cells those multipotent cells may develop.

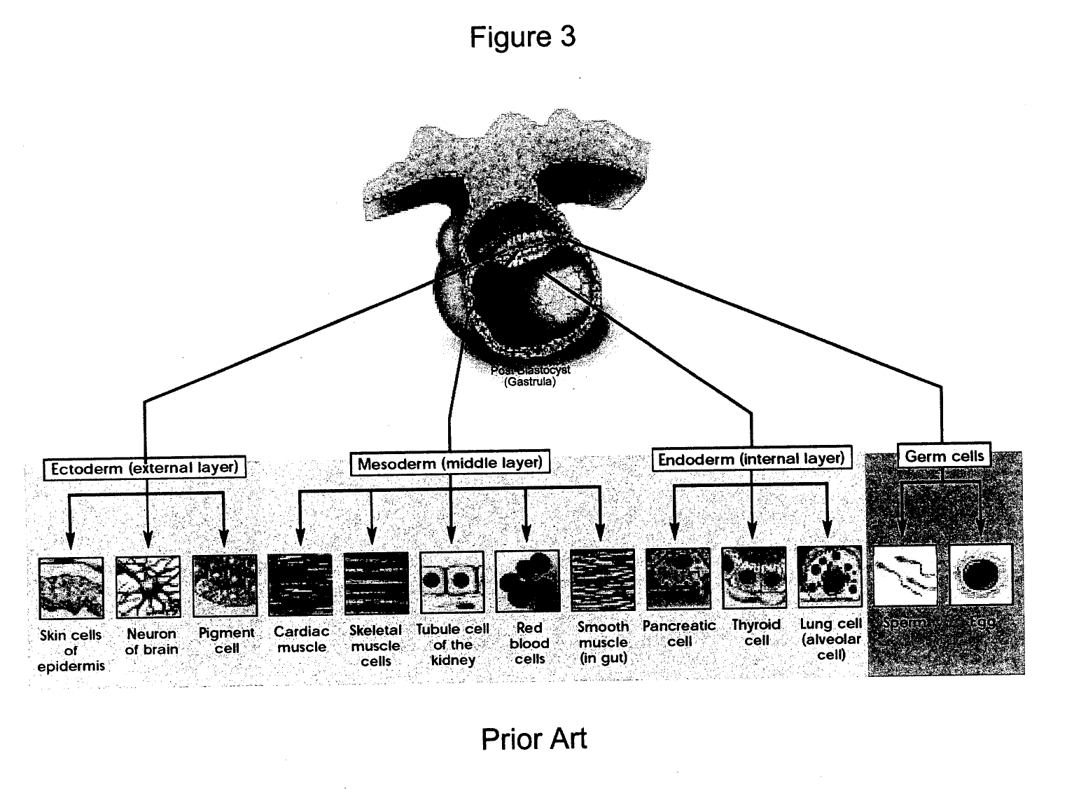

FIG. 4, in accordance with one embodiment of the present invention, shows a method in which a non-human mammalian stem cell is utilized to create a human stem cell.

FIG. 5, in accordance with one embodiment of the present invention, shows a method in which a human oocyte is utilized to create a human stem cell without creating or destroying a human embryo.

FIG. 6, in accordance with one embodiment of the present invention, shows a method to culture a human stem cell (created without creating or destroying a human embryo) to produce stem cell lines.

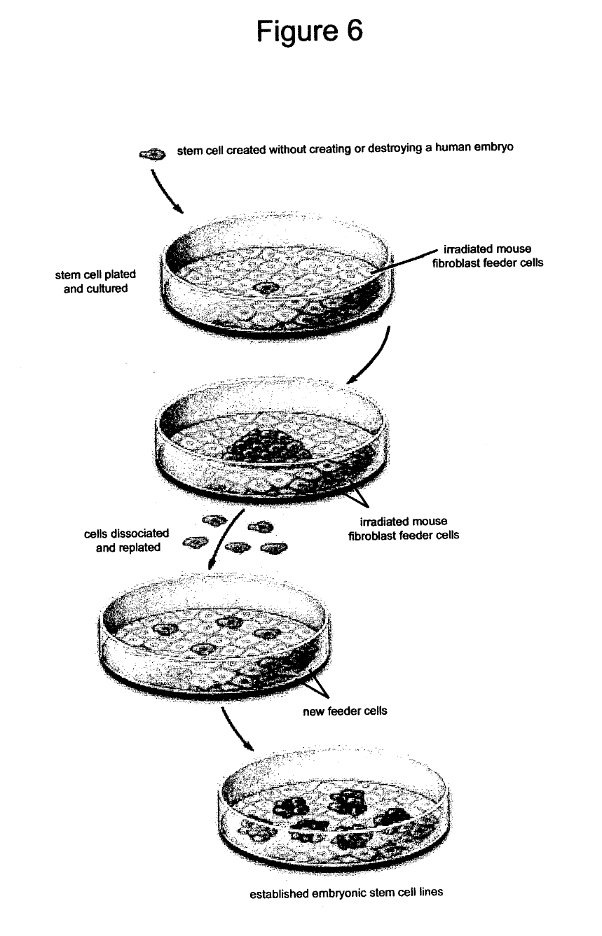

### DETAILED DESCRIPTION

The following definitions are provided for a consistent understanding of the invention described.

The term “blastocyst” refers to the stage of an embryo marked by the existence of a sphere made up of an outer layer of cells (the trophectoderm), a fluid filled cavity (the blastocoel), and a cluster of cells on the interior (the inner cell mass).

The term “donor” refers to any cell whose nucleus is the transferred into an enucleated cell.

The term “enucleated” refers to any cell which has had its nucleus removed.

The term “fetal gonadal tissue” refers to those tissues of a fetus that eventually develop into the sex organs (ovaries, testis, etc.).

The term “mammalian” or “mammal” refers to any animal of the class Mammalia. For example this includes monkeys, chimpanzees, gorillas, cows, buffalo, pigs, sheep, horses, rabbits, guinea pigs, mice, rats, and hamsters.

The term “multipotent stem cell” refers to any cell that has the capability of differentiating into cells with particular functions, or groups of tissues. Multipotent stem cells are more specialized than pluripotent stem cells.

The term “nuclear transfer unit” refers to any cell that is the product of a combination of an enucleated cell and a donor nucleus.

The term “oocyte” refers to an unfertilized egg.

The term “pluripotent stem cell” refers to any cell that has the capability of differentiating into all cells of a developing embryo's body, but in vivo will not differentiate into the trophoblast or those tissues that develop from the trophoblast. For example, the inner cell mass of a blastocyst consists of pluripotent stem cells. Undifferentiated cells from the epiblast are also pluripotent stem cells.

The term “somatic cell” refers to any cell other then sperm cells, egg cells or cells that can become sperm or egg cells. By way of example this term includes any cells obtained from skin, muscle, lung, pancreas, liver and stomach tissues.

The term “stem cell” refers to a cell from the embryo, fetus, or adult that has, under certain conditions, the ability to reproduce itself for long periods or, in the case of adult stem cells, throughout the life of the organism. It also can give rise to specialized cells that make up the tissues and organs of the body.

The term “stem cell line” refers to a group of cells cultured from a stem cell without undergoing further differentiation.

The term “totipotent stem cell” refers to any of the cells of the very early embryo which have the capacity to differentiate into all extra embryonic membranes and tissues, all tissues of the embryo, and all postembryonic tissues and organs.

In the present invention human pluripotent or multipotent stem cells are created without creating or destroying a human embryo. The present invention achieves this by transferring the nucleus of a human somatic donor cell into an enucleated pluripotent or multipotent stem cell of a non-human mammalian source. These stem cells can be cultured and maintained to create human stem cell lines.

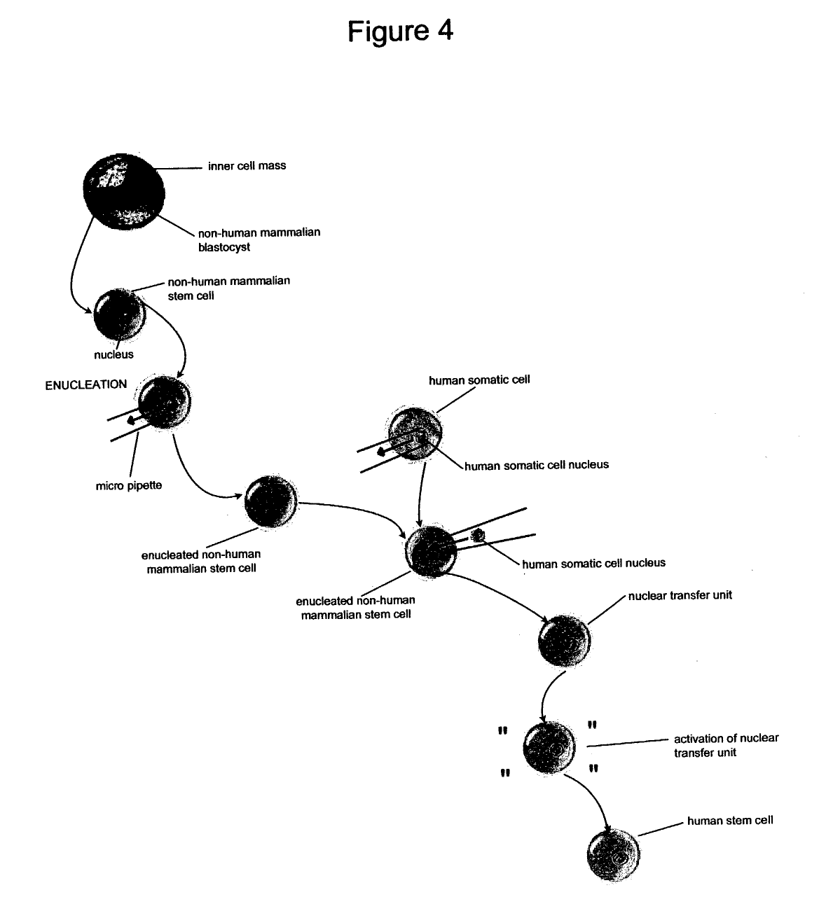

In one aspect of the present invention, non-human mammalian stem cells are obtained. The cells can be obtained from a pre-blastocyst, blastocyst or post-blastocyst embryo. The stem cells (either pluripotent or multipotent) are removed from the embryo by methods known in the art. The cells can then be utilized immediately or multiplied in a culture that retains their pluripotent or multipotent nature. FIG. 4 shows this method utilizing a pluripotent stem cell from the inner cell mass of a non-human mammalian blastocyst.

The nucleus of a stem cell obtained previously is removed forming an enucleated non-human mammalian stem cell. Methods for enculating cells are well known in the art. Typically this is achieved by inserting a needle or micro pipette into a cell and removing the nucleus into the needle or micro pipette. The needle or micro pipette can be removed from the cell without rupturing the plasma membrane. FIG. 4 shows an isolated stem cell being enucleated by a micro pipette.

A somatic cell is obtained from a human somatic donor. The nucleus from the human somatic cell is transferred into the enucleated stem cell (creating a nuclear transfer unit) utilizing nuclear transfer technology known in the art, including fusion or micro injection techniques. By way of example, cell fusion can be achieved by electrofusion. Electrofusion is generally accomplished by placing the enucleated cell and the donor cell adjacent to one another. A pulse of electricity is provided to cause a transient breakdown of the plasma membrane of both cells. Upon reformation, the two membranes intermingle causing small channels to open. These channels enlarge until eventually the two cells (the enucleatated cell and the donor cell) become one. Another fusion method known in the art utilizes a Sendai virus as a fusogentic agent. Another nuclear transfer method known in the art is micro injection. Micro injection can be accomplished by directly injecting the donor cell nucleus into the enucleated cell forming a nuclear transfer unit. FIG. 4 shows the nuclear transfer being accomplished by micro injection.

The nuclear transfer unit is activated by methods known in the art. One example of activation known in the art is achieved by exposing the nuclear transfer unit to a sub-physiological temperature (which may be room-temperature). In another example, activation is accomplished by exposing the nuclear transfer unit to various chemical treatments. Activation may also occur spontaneously.

In another aspect of the present invention, the nuclear transfer unit formed by the combination of an enucleated non-human mammalian stem cell and a human somatic cell is utilized for research and testing of known and new activation methods.

In another aspect of the present invention the non-human mammalian stem cells are obtained from a non-human mammalian source post the blastocyst stage including non-human mammalian stem cells from fetal tissues. Methods for the identification and isolation of post blastocyst stem cells are known in the art and include cells that are obtained from primordial germ cells (destined to become egg or sperm), such as cells from fetal gonadal tissue.

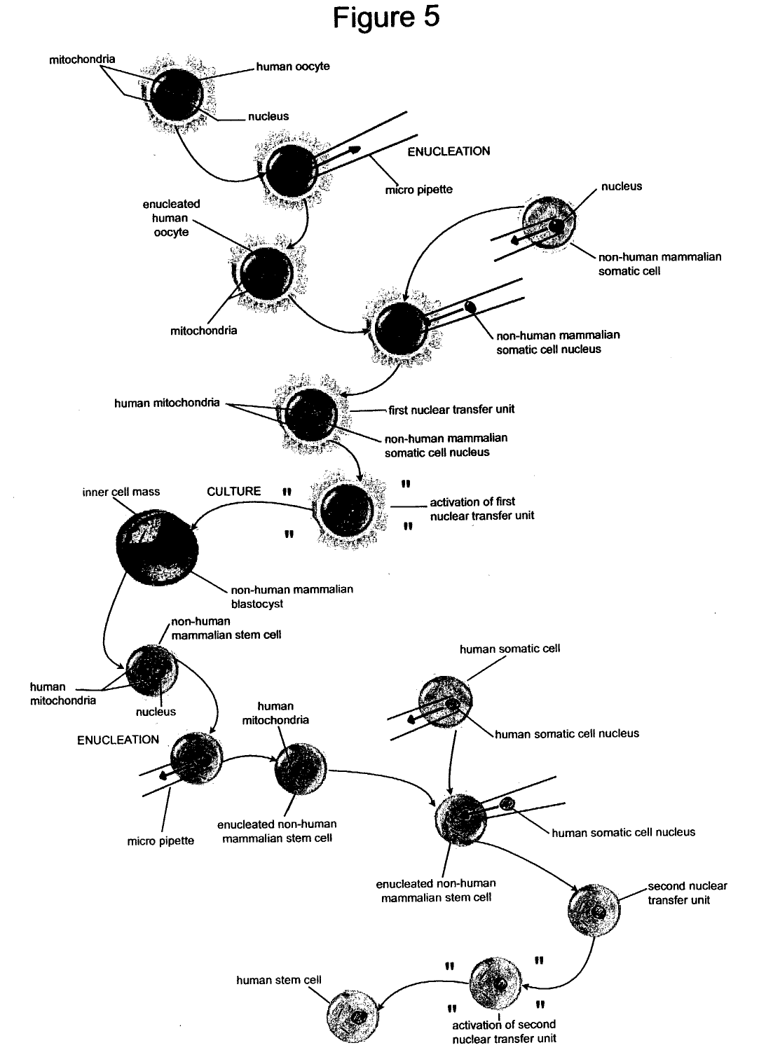

In another aspect of the present invention, illustrated in FIG. 5, a donor human oocyte is obtained. Although not necessary, it is clinically preferable to utilize an oocyte from the closest female relative, following maternal lines, of the human somatic donor (including the human somatic donor herself). This will help ensure compatibility of the nucleus with the cellular mitochondria. Mitochondria are cellular organelles which play important roles in cellular respiration. Mitochondria are passed from one generation to the next through maternal lines. Compatibility of the nucleus with cellular mitochondria will aid in preventing rejection of therapeutic tissues which can be derived from the stem cells and stem cell lines of the present invention.

The nucleus of the human donor oocyte is removed forming an enucleated oocyte. FIG. 5 shows the removal of an oocyte's nucleus by micro pipette. A first somatic cell (which is any cell other then egg or sperm) is obtained from a non-human mammalian source. The nucleus from the first somatic cell is transferred into the enucleated oocyte (creating a first nuclear transfer unit) utilizing nuclear transfer technology. FIG. 5 shows the nucleus from the first somatic cell being transferred into the enucleated oocyte by micro injection. The first nuclear transfer unit is activated.

The activated first nuclear transfer unit is then cultured to a pre-blastocyst, blastocyst or post-blastocyst embryo until the development of stem cells (either pluripotent or multipotent). FIG. 5 shows the embryo at the blastocyst stage. Because the nuclear genetic material of the developing embryo is derived from a non-human mammalian source, the resulting embryo is non-human. However, since a human oocyte was used, the embryo's cells contain at least some mitochondria identical to the human oocyte source.

The stem cells are removed from the embryo. The stem cells can then be utilized immediately or multiplied in a culture that retains their pluripotent or multipotent nature. The nucleus of a stem cell obtained previously is removed forming an enucleated stem cell. FIG. 5 shows the enucleation procedure being accomplished by micro pipette. A second somatic cell is obtained from a human somatic donor. The nucleus from the second somatic cell is transferred into the enucleated stem cell (creating a second nuclear transfer unit) utilizing nuclear transfer technology. FIG. 5 shows the nuclear transfer being accomplished by micro injection. The second nuclear transfer unit is then activated.

In another aspect of the present invention, the nuclear transfer unit formed by the combination of an enucleated human oocyte and a non-human somatic cell is utilized for research and testing of known and new activation methods. In another aspect of the present invention, human stem cells are created that can be used in researching or treating ailments caused by mitochondrial defects.

Mitochondrial defects have been linked to certain diseases such as Parkinson's disease and Huntington's disease. These defects can originate ancestrally and be passed heretically from an individual's mother. Other defects can occur in an individual's mitochondria from mutations during the individual's lifetime (including mutations occurring prior to birth). To the extent mitochondrial defects are caused by mutations in a given individual, one aspect of the present invention teaches a method where non-defective mitochondria (that is identical or nearly identical to the individual's mitochondria prior to mutation) can be passed to stem cells for eventual therapeutic uses in that individual.

To the extent mitochondrial defects are hereditary in a given individual; one aspect of the present invention teaches a method where healthy mitochondria can be passed to stem cells for eventual therapeutic uses in that individual.

In another aspect of the present invention the human stem cells created by the preceding methods are cultured in a medium to preserve their pluripotent or multipotent nature thereby yielding stem cell lines. Culturing mediums and methods to create stem cell lines are known in the art. By way of example, and illustrated in FIG. 5, one method known in the art to generate human stem cells lines involves taking isolated stem cells and plating them in culture dishes containing growth medium supplemented with fetal bovine serum on feeder layers of mouse embryonic fibroblasts that had been gamma-irradiated to prevent replication. After multiple divisions of the cultured stem cells, the cultured stem cells are dissociated from one another and replated in the same culture conditions. Colonies of apparently homogeneous cells can then be selectively removed, mechanically dissociated, and replated.

While the invention has been described with respect to a limited number of embodiments, those skilled in the art, having benefit of this disclosure, will appreciate that other embodiments can be devised which do not depart from the scope of the invention as disclosed here. Accordingly, the scope of the invention should be limited only by the attached claims.

What is claimed is:

1. A method of creating a human stem cell, comprising:
(a) transferring the nucleus of a human somatic donor cell into an enucleated non-human mammalian stem cell forming a nuclear transfer unit; and
(b) activating the nuclear transfer unit.

2. A method as claimed in claim 1, where the non-human mammalian stem cell is pluripotent.

3. A method as claimed in claim 1, where the non-human mammalian stem cell is multipotent.

4. A method as claimed in claim 1, further comprising:
(c) culturing the human stem cell to yield a human stem cell line.

5. A method of creating a human stem cell, comprising:
(a) enucleating a non-human mammalian stem cell; and
(b) transferring the nucleus of a human somatic donor cell into the enucleated non-human mammalian stem cell.

6. A method as claimed in claim 5, where the non-human mammalian stem cell is pluripotent.

7. A method as claimed in claim 5, where the non-human mammalian stem cell is multipotent.

8. A method as claimed in claim 5, further comprising:
(c) culturing the human stem cell to yield a human stem cell line.

9. A method of creating a human stem cell, comprising:
(a) enucleating a non-human mammalian stem cell;
(b) transferring the nucleus of a human somatic donor cell into the enucleated non-human mammalian stem cell forming a nuclear transfer unit; and
(c) activating the nuclear transfer unit.

10. A method as claimed in claim 9, where the non-human mammalian stem cell is pluripotent.

11. A method as claimed in claim 9, where the non-human mammalian stem cell is multipotent.

12. A method as claimed in claim 9, further comprising:
(d) culturing the human stem cell to yield a human stem cell line.

13. A method of creating a human stem cell, comprising:
(a) enucleating a human oocyte;
(b) transferring the nucleus of a non-human mammalian somatic donor cell into the enucleated human oocyte creating a first nuclear transfer unit;
(c) activating the first nuclear transfer unit.

14. A method as claimed in claim 13 further comprising:
(d) culturing the first nuclear transfer unit to yield at least one non-human stem cell;

15. A method as claimed in claim 13 further comprising:
(d) culturing the human stem cell to yield a human stem cell line.

16. A method of creating a human stem cell; comprising:
(a) enucleating a human oocyte;
(b) transferring the nucleus of a non-human mammalian somatic donor cell into the enucleated human oocyte creating a first nuclear transfer unit;
(c) activating the first nuclear transfer unit;
(d) culturing the first nuclear transfer unit to yield at least one non-human stem cell;
(e) enucleating the non-human stem cell; and
(f) transferring the nucleus of a human somatic donor cell into the enucleated non-human stem cell to create a second nuclear transfer unit;

17. A method as claimed in claim 16 where the non-human stem cell is pluripotent.

18. A method as claimed in claim 16 where the non-human stem cell is multipotent.

19. A method as claimed in claim 16 further comprising:
(g) culturing the human stem cell to yield a human stem cell line.

20. A method of creating a human stem cell, comprising:
(a) enucleating a human oocyte;
(b) transferring the nucleus of a non-human mammalian somatic donor cell into the enucleated human oocyte creating a first nuclear transfer unit;
(c) activating the first nuclear transfer unit;
(d) culturing the first nuclear transfer unit to yield at least one non-human stem cell;
(e) enucleating the non-human stem cell;
(f) transferring the nucleus of a human somatic donor cell into the enucleated non-human stem cell to create a second nuclear transfer unit; and
(g) activating the second nuclear transfer unit.

21. A method as claimed in claim 20 where the non-human stem cell is pluripotent.

22. A method as claimed in claim 20 where the non-human stem cell is multipotent.

23. A method as claimed in claim 20 further comprising:
(h) culturing the human stem cell to yield a human stem cell line.

24. A method of creating a human stem cell, comprising:
(a) step for transferring the nucleus of a human somatic donor cell into an enucleated non-human mammalian stem cell forming a nuclear transfer unit; and
(b) step for activating the nuclear transfer unit.

25. A method of creating a human stem cell, comprising:
(a) step for enucleating a non-human mammalian stem cell; and
(b) step for transferring the nucleus of a human somatic donor cell into the enucleated non-human mammalian stem cell.

26. A method of creating a human stem cell, comprising:
(a) step for enucleating a human oocyte;
(b) step for transferring the nucleus of a non-human mammalian somatic donor cell into the enucleated human oocyte.

27. Method for creating a nuclear transfer unit comprising:
(a) enucleating a non-human mammalian stem cell; and
(b) transferring the nucleus of a human somatic donor cell into the enucleated non-human mammalian stem cell.

28. Method for creating a nuclear transfer unit comprising:
(a) step for enucleating a non-human mammalian stem cell; and
(b) step for transferring the nucleus of a human somatic donor cell into the enucleated non-human mammalian stem cell.

29. Method for creating a nuclear transfer unit comprising:
(a) enucleating a human oocyte; and
(b) transferring the nucleus of a non-human mammalian somatic donor cell into the enucleated oocyte.

30. Method for creating a nuclear transfer unit comprising:
(a) step for enucleating a human oocyte; and
(b) step for transferring the nucleus of a non-human mammalian somatic donor cell into the enucleated oocyte.
